(19) United States
(12) Patent Application Publication
(10) Pub. No.: US 2025/0325176 A1
(43) Pub. Date: Oct. 23, 2025

(54) INFANT INTRAORAL IMAGING DEVICE

(71) Applicants: Diana Alatalo, Worcester, MA (US); Kenza Bezzat, Woburn, MA (US); Nicolas Loycano, Bridgewater, MA (US); Jacob McDonald, Salem, NH (US); Samantha Turner, Morganville, NJ (US); Haichong Zhang, Shrewsbury, MA (US)

(72) Inventors: Diana Alatalo, Worcester, MA (US); Kenza Bezzat, Woburn, MA (US); Nicolas Loycano, Bridgewater, MA (US); Jacob McDonald, Salem, NH (US); Samantha Turner, Morganville, NJ (US); Haichong Zhang, Shrewsbury, MA (US)

(21) Appl. No.: 19/184,332

(22) Filed: Apr. 21, 2025

Related U.S. Application Data
(60) Provisional application No. 63/636,297, filed on Apr. 19, 2024.

(51) Int. Cl. A61B 1/24; A61B 1/00; A61C 9/00

(52) U.S. Cl. CPC A61B 1/24; A61B 1/00147; A61C 9/0053; A61B 2503/04

(57) ABSTRACT

A targeted imaging device can be inserted into a small oral cavity such as found in infants and the oral cavity of individuals unable to follow directions to obtain 2D or 3D images of the upper palate and gums or teeth. The intraoral imaging device positions an imaging element in an oral cavity of an infant, and probes successive fields of view for capturing a series of adjacent images in the oral cavity. A transparent, resilient tip is resistant to moisture and abrasion or discomfort which could result from a hard, sharp or rigid insertion. Captured images depict the palate and gumline, and aggregation or synthesis of consecutive fields of view allows reconstruction of a continuous image via photogrammetry. The reconstructed image is invoked for fabrication of a corrective appliance, and can be particularly beneficial for fabrication of a presurgical nasoalveolar molding often preceding surgical treatment for a cleft palate.

### RELATED APPLICATIONS

This patent application claims the benefit under 35 U.S.C. § 119(e) of U.S. Provisional Patent App. No. 63/636,297, filed Apr. 19, 2024, entitled “INFANT INTRAORAL IMAGING DEVICE,” incorporated herein by reference in entirety.

### BACKGROUND

In a healthy individual, fundamental functions like breathing and eating are innate tasks often taken for granted. However, for babies born with a cleft palate, these considerably simple acts become challenging. Breathing is a conscious effort and the process of feeding without choking is problematic because of the gaping hole in the roof of the infant's mouth. Other health issues associated with cleft palate include hearing loss, speech delays, problems with teeth development and predisposition to different types of infections. When left untreated, an infant's nourishment is directly impacted, preventing healthy growth and development.

### SUMMARY

A targeted imaging device can be inserted into a small oral cavity such as found in infants and the oral cavity of individuals unable to follow directions to obtain 2D or 3D images of the upper palate and gums or teeth. The intraoral imaging device positions an imaging element in an oral cavity of an infant, and probes successive fields of view for capturing a series of adjacent images in the oral cavity. A transparent, resilient tip is resistant to moisture and abrasion or discomfort which could result from a hard, sharp or rigid insertion. Captured images depict the palate and gumline, and aggregation or synthesis of consecutive fields of view allows reconstruction of a continuous image via photogrammetry. The reconstructed image is invoked for fabrication of a corrective appliance, and can be particularly beneficial for fabrication of a presurgical nasoalveolar molding (NAM) often preceding surgical treatment for a cleft palate.

Oral abnormalities in newborns can negatively impact breastfeeding success and may require medical intervention. Identifying and repairing oral abnormalities requires clear visualization of the area and may require creating a mold of the oral cavity either in whole or in part. Visualization of an infant's oral cavity is difficult due to the small size and the infant's inability to follow directions and hold their mouth open on demand. While large abnormalities such as a cleft palate are often easy to identify, smaller abnormalities may not be readily apparent, such as incomplete uvula and high-arched palate. Creating a mold of the oral cavity in infants that enables creating a 3-dimensional image is dangerous as infants may aspirate the molding materials.

Configurations herein are based, in part, in the observation that treatment of a cleft palate for newborns is often of limited accessibility in underdeveloped areas. Cleft palate procedures are often preceded by temporary implantation of the NAM for repositioning anatomical features in advance of surgery. Unfortunately, conventional approaches to cleft palate treatment, and of the NAM in particular, suffer from the shortcoming of repetitive visits to a medical facility as well as discomfort to the infant patient from oral examinations for gathering, measuring and sizing the needed anatomical parameters for NAM fabrication. Patients in remote areas may be unable to bear the travel and/or expense to properly develop the NAM as a surgical precursor.

Accordingly, configurations herein substantially overcome the shortcomings of infant oral imaging, and in particular imaging for NAM fabrication, by providing an intraoral imaging device having a resilient imaging tip for insertion into an infant oral cavity, safely manipulating at a safe insertion depth for imaging a series of consecutive fields of view (FOV) around the oral cavity for reconstructing a complete 3-dimensional oral image suitable for fabrication of a NAM appliance fitted to the infant patient. Image capture occurs by a local connected device, and may be transmitted or downloaded to a fabrication facility for generating the fitted NAM device. The resilient imaging tip has a rounded tip resistant to infant gum movement, and simulates a feeding nipple, mitigating discomfort to the infant patient. A transparent material or windowed aperture permits image capture from the resilient tip as it traverses the oral cavity for capturing the needed FOV positions for the series of images for reconstruction.

In further detail, an oral imaging device as disclosed herein includes a probe tip configured for insertion into an oral cavity, where the probe tip has an interior bore and a length based on an insertion depth, and a probe base, such that the probe tip extends from the probe base and has a wider cross section than the probe tip for preventing oral insertion beyond the probe tip. An imaging sensor is disposed into the interior bore for capturing a series of images of the oral cavity, and a handle is attached to the probe base for manipulation of the probe tip and imaging sensor within the oral cavity.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a side schematic view of an oral imaging device as disclosed herein;

FIG. 2 shows a system for image capture and processing using the device of FIG. 1;

FIG. 3 shows a perspective view of a configuration of an oral imaging device as in FIG. 1; and

FIG. 4 shows an exploded view of an alternate configuration of the oral imaging device.

### DETAILED DESCRIPTION

Depicted below are example configurations of an infant intraoral imaging device for imaging an oral cavity of an infant or other non-conversant patient for comprehensive 360° imaging with a minimally invasive imaging probe emulating a feeding nipple. The use of a resilient, soft material results in a similar sensation to a comforting feeding nipple to keep the infant patient calm and relaxed. Positioning/manipulation of the inserted device, along with rotation of the imaging sensor, provides a series of images from successive fields of view to allow reconstruction of a 3-dimensional image for fabrication of a corrective appliance beneficial to treatment of the cleft palate condition.

This structural birth defect is a result of the incomplete fusion of the roof of the infant's mouth during pregnancy. Cleft palates can range in shape and size with the most severe cases starting in the back of the mouth, splitting the upper palate from the uvula reaching until the front upper lip and into the nostril. Corrective surgical intervention is necessary to close the gap and restore the normal anatomy. Before undergoing the procedure, a presurgical nasoalveolar molding or NAM treatment is made to prepare the infant for surgery. The intraoral molding plate is often accompanied by a nasal stent to support any nostril deformities. The purpose of the presurgical NAM is to lessen the severity of the cleft by pushing the gap together to facilitate surgical efforts.

In order to build the NAM device, an impression mold of the oral cavity is first captured to model the cleft. The conventional molding process is highly invasive and can be very traumatic for newborns with cleft palate. In addition to the extensive costs associated with the corrective therapy, routine fittings and remolding for adjustments call for frequent in person visits. In regions where NAM treatment is not easily accessible nor affordable, babies born with cleft palate often suffer the consequences of an unrepaired palate. Malnourishment as a result of this medical disparity drives high mortality rates in early infanthood. Configurations herein facilitate the NAM fabrication procedure so that needful infants can receive lifesaving treatment and lead a better, healthier quality of life.

Conventional approaches for assessing the oral cavity of infant patients with cleft palate require an invasive, globally inaccessible and inconvenient impression molding technique, which is often impractical in remote and/or underdeveloped regions. To remedy this impediment, approaches disclosed herein present a noninvasive, sustainable, and accessible device that can digitally capture accurate 3D images of the oral cavity to create a computer-aided design (CAD) model, eliminating the manual component and reducing the frequency of in person hospital visits and improving the patient experience. The disclosed approach allows specialists to remotely evaluate and facilitate the 3D printing of nasoalveolar molding, accelerating the process of treatment and therefore increasing opportunities to receive surgery.

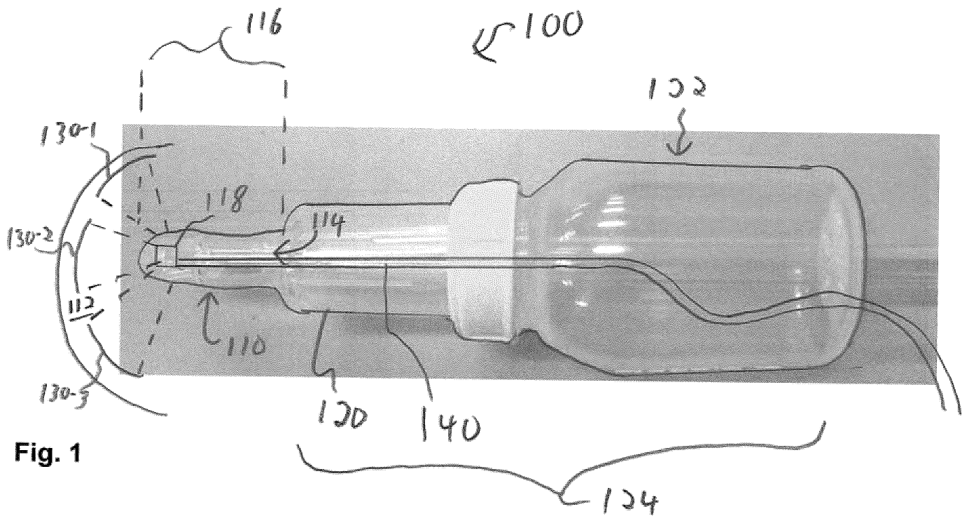

FIG. 1 is a side schematic view of an oral imaging device as disclosed herein. In general, oral imaging uses one or more cameras to capture images when the oral cavity is open. The camera may take a single image or multiple images either as a series of still images or as a video. A series of images taken from different angles may be matched to compose a 3-dimensional image of an object such as a tooth. Various methods exist to obtain a 3-dimensional image from 2-dimensional images using either single or multiple cameras. These conventional methods generally expect the oral cavity to remain open and the patient to be able to follow directions from the individual taking the images. Additionally, the camera(s) must maintain a distance between the lens and the object of interest to allow incident light to illuminate the object. Infants and individuals unable to follow directions cannot use conventional oral imaging devices.

Referring to FIG. 1, the oral imaging device 100 includes a probe tip 110 configured for insertion into an oral cavity 112, such that the probe tip has an interior bore 114 and a length 116 based on an insertion depth. The probe tip 110 extends from a probe base 120, where the probe base 120 has a wider cross section than the probe tip and is configured for preventing oral insertion into the oral cavity 112 beyond the probe tip 110. In one configuration, the probe tip 110 and base 120 simulate an infant feeding nipple for familiarity and acceptance by the infant patient. An imaging sensor 118 resides in the interior bore 114, and a handle 122 or shaft attaches to the probe base 120 for manipulation of the probe tip 110 and imaging sensor 118 within the oral cavity 112.

In an example configuration, the probe tip 110 is a transparent, resilient material, such that imaging sensor 118 is operable for reception through the transparent material for gathering images of multiple fields of view 130-1 . . . 130-N (130 generally) around the oral cavity 112. Over several positioning iterations by the handle 122, the plurality of images define adjacent and overlapping fields of view of the oral cavity.

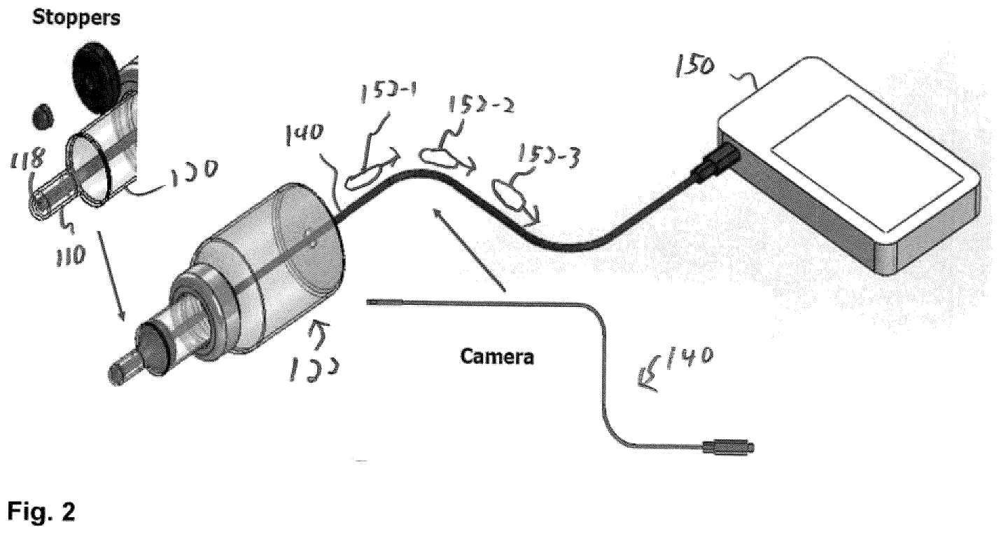

FIG. 2 shows a system for image capture and processing using the device of FIG. 1. Referring to FIGS. 1 and 2, the handle 122 defines a hand-held housing unit 124 that provides a stabilizing structure for a flexible insertion tube imaging scope 140, terminating in the imaging sensor 118. The housing 124 allows the imager to control the depth of the tip 110 into the oral cavity 112 and to physically rotate the camera or image sensor 118 for manual control. The housing 124 contains an adjustable stopper at the base to prevent the scope from being inserted beyond the posterior of the oral cavity. As the probe base 120 has a diameter greater than a diameter of the probe tip 110, the diameter of probe base is therefore positioned based on an oral cavity 112 size for limiting the insertion depth of the probe tip 110 based on the length 116.

The imaging sensor 118 connects to an imaging processor 150 via a cable or tether of the imaging scope 140, such that the imaging processor 150 is configured to receive a plurality of images 152-1 . . . 152-N (152 generally) of the oral cavity 112, and execute instructions for photogrammetry aggregation of the plurality of images into a continuous imaged region representative of the oral cavity 112 in 2-dimensional or 3 dimensional form, depending on the imaging medium and reconstruction of the series of images 152-N.

The imaging processor 150, which may include various storage and network connected transport mediums, is configured to gather the images 152 indicative of a palate and a gumline of the oral cavity. In an example configuration, the imaging processor 150 computes, from the gathered images, parameters for a corrective oral appliance such as the NAM. In one configuration, the imaging processor 150 is configured to gather photolithography based images 152 from the oral cavity 112.

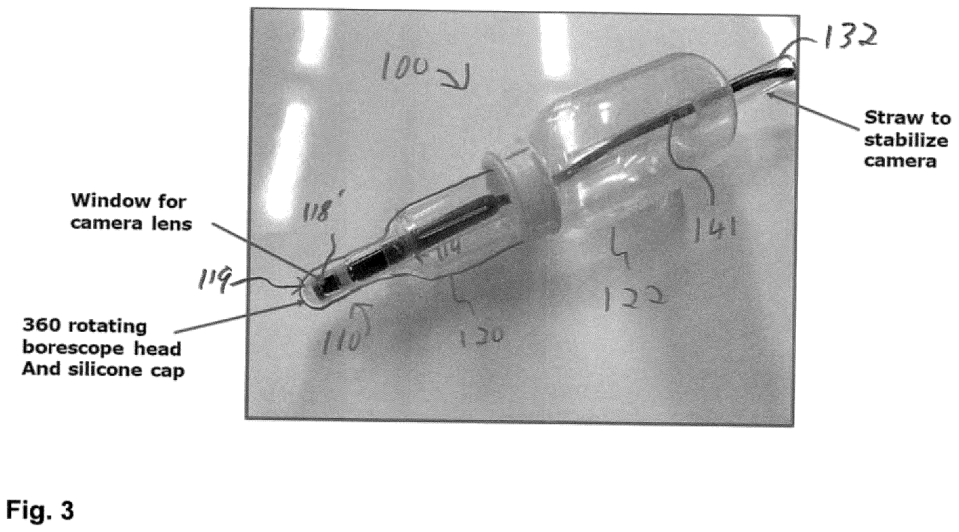

FIG. 3 shows a perspective view of a configuration of an oral imaging device 100 as in FIG. 1. The disclosed approach allows for imaging in the oral cavity 112 while the patient's mouth is closed and the structures of the oral cavity are in contact with the device. The device houses the camera(s) end of an imager such as endoscope or boroscope, hereafter referred to as “scope”, such that the camera or similar imaging sensor 118 can be rotated to obtain images of different oral structures. The device 100 includes a hygienic soft tip 110 that encases the tip of the scope and can either be sanitized, sterilized, or disposed of between patients depending upon material. The tip 110 provides protection of the oral cavity and produces a gap at an appropriate depth to allow any lighting used by the scope to illuminate the structures of interest for imaging. The tip 110 is preferably transparent, however depending on the optical clarity of the tip, a hole for the scope's camera can be made if the scope is waterproof. The tip allows the patient to suck on it, which action is soothing and a natural reflex for an infant. The scope's camera can be moved around the oral cavity 112 to image any structure of interest as the tip protects the scope from chewing actions common to infants. Alternatively, other sensory mediums, such as ultrasound, may be employed. The scope need not be permanently integrated into the device 100, but may be a standalone implementation with an elongated body, imaging sensor 118 at a distal end, and connecting cables 141, sized for insertion through the handle 122 and bore 114.

In this manner, the imaging sensor 118 may take the form of a rotating sensing head 118′, such that the rotational head 118′ is configured for rotation within the probe tip 110, where rotation provides a selection of a field of view 130 of the imaging sensor. The imaging sensor 118 is resistant to liquid and the probe tip further includes a window gap 119 defined by an aperture in the probe tip, such that the imaging sensor 118 is aligned with the window gap 119 for image reception. A rearward tube 132 provides a secondary support for a wire or tether 141 powering the imaging sensor 118 and receiving the images 152.

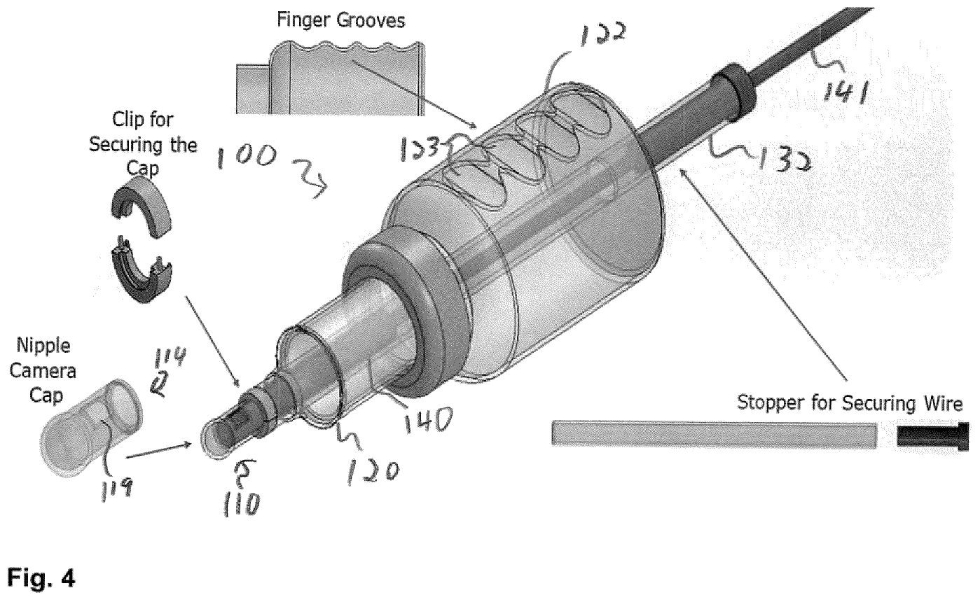

FIG. 4 shows an exploded view of an alternate configuration of the oral imaging device 100 with ergonomic and assemblage enhancements. In an example configuration and field of use, the device 100 provides a method for forming a corrective oral appliance as a preliminary prophylactic step in anticipation of a cleft palate surgical repair, particularly for an infant patient. The approach disposes the probe tip 110 into the oral cavity 112 of a patient, where the probe tip includes the interior bore 114 housing the imaging sensor 118, which is part of a scope 140 or imaging device having a cable 141 for interoperability with the imaging processor 150 as described above. A technician, operator or examination sequence orients the probe tip 110 within the oral cavity 112 for gathering a series of consecutive field-of-view (FOV) 130 positions of the oral cavity 112. For the aggregate positions or iterations, the imaging sensor 118 transmits the plurality of images 152 corresponding to the consecutive FOV positions to the imaging processor 150. The imaging processor 150 computes, from photogrammetry processing of the consecutive FOV positions, an indication of a developmental deviation of palate and a gumline by assembling a 3-dimensional view of model including the problematic deformations. This model allows generation, based on the indication of developmental deviation, specifications for a corrective appliance such as the MAM in preparation for a full corrective procedure to remedy the cleft condition.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. An oral imaging device, comprising:
a probe tip configured for insertion into an oral cavity, the probe tip having an interior bore and a length based on an insertion depth;
a probe base, the probe tip extending from the probe base, the probe base having a wider cross section than the probe tip and configured for preventing oral insertion beyond the probe tip;
an imaging sensor inserted into the interior bore; and
a handle, the handle attached to the probe base for manipulation of the probe tip and imaging sensor within the oral cavity.

2. The device of claim 1 wherein the imaging sensor further comprising a rotating sensing head, the rotational head configured for rotation within the probe tip, the rotation providing selection of a field of view of the imaging sensor.

3. The device of claim 1 wherein the probe tip is a transparent, resilient material, the imaging sensor operable for reception through the transparent material.

4. The device of claim 1 wherein the imaging sensor is resistant to liquid and the probe tip further comprises a window gap defined by an aperture in the probe tip, the imaging sensor aligned with the window gap for image reception.

5. The device of claim 1 wherein the probe base has a diameter greater than a diameter of the probe tip, the diameter of probe base based on an oral cavity size for limiting an insertion depth of the probe tip.

6. The device of claim 1 wherein the imaging sensor connects to an imaging processor, the imaging processor configured to:
receive a plurality of images of the oral cavity; and
execute instructions for photogrammetry aggregation of the plurality of images into a continuous imaged region.

7. The device of claim 6 wherein the plurality of images define adjacent and overlapping fields of view of the oral cavity.

8. The device of claim 6 wherein the imaging processor is configured to gather images indicative of a palate and a gumline of the oral cavity; and further configured to compute, from the gathered images, parameters for a corrective oral appliance.

9. The device of claim 1 wherein the imaging sensor is configured to gather photolithography based images from the oral cavity.

10. A method for forming a corrective oral appliance, comprising:
disposing a probe tip into an oral cavity of a patient, the probe tip including an interior bore housing an imaging sensor;
orienting the probe tip within the oral cavity for gathering a series of consecutive field-of-view (FOV) positions of the oral cavity;
transmitting a plurality of images corresponding to the consecutive FOV positions to an imaging processor;
computing, from photogrammetry processing of the consecutive FOV positions, an indication of a developmental deviation of palate and a gumline; and
generating, based on the indication of developmental deviation, specifications for a corrective appliance.
